(19) United States
(12) Patent Application Publication
(10) Pub. No.: US 2007/0065893 A1
(43) Pub. Date: Mar. 22, 2007

(54) Liquid-phase galactose oxidase-schiff's assay

(76) Inventors: Carol Carte, Cayuga (CA); Mike Evelegh, Dundas (CA); Robert Zawydiwski, Stoney Creek (CA)

Correspondence Address: BERESKIN AND PARR, 40 KING STREET WEST, BOX 401, TORONTO, ON, M5H 3Y2, CA

(21) Appl. No.: 11/523,057

(22) Filed: Sep. 19, 2006

Related U.S. Application Data
(60) Provisional application No. 60/717,758, filed on Sep. 19, 2005.

(51) Int. Cl. C12Q 1/34; C12Q 1/26

(52) U.S. Cl. 435/18; 435/25

(57) ABSTRACT

The invention provides an improved method for detecting cancer or a precancerous condition in a sample using an oxidation agent, such as galactose oxidase, and an aldehyde detection agent, such as Schiff's reagent that does not require the sample to be immobilized onto a solid support. The invention also provides kits comprising the components necessary for carrying out the methods of the invention.

### FIELD OF THE INVENTION

The invention relates to an improved method for detecting cancer or a precancerous condition using an oxidation agent and an aldehyde detection agent, such as galactose oxidase and Schiff's reagent. The invention also relates to kits comprising the components necessary for carrying out the methods of the invention.

### BACKGROUND OF THE INVENTION

The galactose oxidase-Schiff's (GOS) test is used to detect carbohydrate markers (e.g. D-galactose [Gal] or N-acetyl-D-galactosamine [GalNAc] or D-galactose-β-[1→3]-N-acetyl-D-galactosamine [GalGalNAc] disaccharide [T or Thomsen-Friedenreich(TF) antigen]) on mucin glycoproteins expressed in cancer or pre-cancerous lesions. These markers may be found in rectal mucus (colon cancer), saliva or sputa (oral, lung cancer), nipple aspirate fluids (breast cancer), other mucous secretions (vaginal fluid [cervical, uterine, endometrial cancer], semen [prostate cancer]) and blood (various cancers).

Shamsuddin (U.S. Pat. No. 5,348,860) teaches a method for detection of the markers involving the adsorption of mucus sample onto a protein-capturing, water-insoluble substrate (e.g., membrane filter) prior to processing by GOS. The prerequisite for sample immobilisation prior to processing by GOS has several limitations: 1) a drying period to permit retention of clinical specimen, 2) rinsing after incubation with galactose oxidase (GO) and more extensive washing after incubation with Schiff's reagent to remove excess enzyme and color developer, respectively, as well as to reduce background due to non-specific reaction products, 3) manual processing of adsorbed specimens, and 4) subjective visual interpretation of results. An additional limitation of the prior art is the use of periodic acid-Schiff's after the GOS procedure to detect glycoproteins and verify the presence of the sample on the solid-phase.

U.S. patent application Ser. No. 10/877,737 teaches a method for generating quantitative or semi-quantitative results based on color attributes such as hue and/or chroma and, in part, mitigates a major disadvantage of visual scoring, i.e., the need for a skilled and experienced analyst. The procedure however, is based on reflectance spectrophotometry and algorithms calculated from information derived from multiple readings within the visible range of the light spectrum. The analysis requires a specialized instrument not commonly available in the diagnostic community.

Accordingly, there is a need in the art to improve the method for detecting cancer using the GOS assay.

### SUMMARY OF THE INVENTION

The present invention provides an improved method for detecting cancer or precancerous conditions in a subject using an oxidation agent and aldehyde detection agent, such as galactose oxidase and Schiff's reagent, that does not require immobilization of the sample from the subject onto a solid support. Instead, the present invention provides an improved method in which samples can be directly reacted with an oxidation agent and aldehyde detection agent in a liquid system without immobilization of the samples onto a solid phase. Additionally, the procedure incorporates a positive control reagent, such as guar, to ensure the activity of the oxidation agent and aldehyde detection agent.

The present invention has several advantages over the prior art. The present invention permits treatment of the sample with the oxidation agent and aldehyde detecting agent, such as galactose oxidase and Schiff's reagent, directly in a liquid system. For instance, the present invention allows chemical disruption/dispersion of a gelled sample, such as sputum to ensure miscibility with the oxidation agent, such as galactose oxidase. The present invention has the advantage over the membrane-based assay in that significantly less time is required to perform the test. Thus, the present invention has the advantage of reduced assay turnaround time. In addition, the method of the present invention provides an objective, semi-quantitative measure of results based on absorption at a predefined wavelength determined with standard laboratory spectrophotometers or microplate readers as compared to a visual non-objective interpretation of the results. The present invention has the advantage that it is amenable to automation and batch processing for high-throughput screening. This is particularly advantageous for population screening. For instance, entire populations or subsets of populations can be easily screened for cancer or a precancerous condition using the method of the invention. In addition, the method of the present invention allows the samples to be pre-measured to remove uncertainty regarding potential false-negatives due to lack of immobilized specimen and obviate the need for post-test treatment (e.g., periodate oxidation followed by Schiff's) to verify presence of sample. The method of the present invention has the potential for better clinical performance.

Accordingly, one aspect of the invention is a method for detecting cancer or a precancerous condition in a subject, wherein a sample from the subject is assayed for the presence of a carbohydrate marker present in the sample associated with cancer or precancerous cells, comprising the steps:

(a) providing a sample from a subject;

(b) mixing the sample with an oxidation agent that is capable of oxidizing susceptible C-6 hydroxyl groups on the carbohydrate markers to aldehydes;

(c) adding an aldehyde detection agent to the mixture that produces a calorimetric change in the presence of an aldehyde; and

(d) detecting the calorimetric change in a liquid system, wherein the colorimetric change produced by the aldehyde detecting reagent is indicative of the presence of a carbohydrate marker associated with cancer or precancerous cells.

Another aspect of the invention is a method for detecting cancer or a precancerous condition in a subject, wherein a sample from the subject is assayed for the presence of a carbohydrate marker present in the sample associated with cancer or precancerous cells, comprising the steps:

(a) mixing the sample with an oxidation agent that is capable of oxidizing susceptible C-6 hydroxyl groups on the carbohydrate markers to aldehydes;

(b) adding an aldehyde detection agent to the mixture that produces a calorimetric change in the presence of an aldehyde; and

(c) detecting the calorimetric change in a liquid system, wherein the calorimetric change produced by the aldehyde detecting reagent is indicative of the presence of a carbohydrate marker associated with cancer or precancerous cells.

In one embodiment, the oxidation agent is galactose oxidase. In another embodiment, the aldehyde detection agent is Schiff's reagent. In a further embodiment, the oxidation agent is galactose oxidase and the aldehyde detection agent is Schiff's reagent.

Another aspect of the invention is a kit for detecting cancer or a precancerous condition, comprising an oxidation agent, such as galactose oxidase, and an aldehyde detection agent, such as Schiff's reagent, and instructions for use according to the method of the invention.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a comparison of the Receiver-Operator Characteristic (ROC) curves for the membrane (Chroma: upper panel) and liquid-phase (A₅₅₀: lower panel) GOS assays, showing the clinical performance (sensitivity and specificity) of each test at various cutoffs with lung sputa from patients diagnosed with cancer (n=12) vs those without cancer (n=8; no pathology, healthy smoker, benign lung disease). Area under the curve (AUC) and statistical significance (p-value) are indicated.

FIG. 2 shows scattergrams for lung sputa obtained from individual subjects and tested in the membrane and liquid-phase GOS formats. Dotted lines indicate cutoffs yielding zero false-positive results.

### DETAILED DESCRIPTION OF THE INVENTION

The present invention provides an improved method for detecting cancer or precancerous conditions in a subject using an oxidation agent and an aldehyde detection agent, such as galactose oxidase and Schiff's reagent. Specifically, the present invention provides an improved method in which samples can be reacted directly with an oxidation agent and an aldehyde detection agent in a liquid system without immobilization of the samples onto a solid phase.

The inventors have compared their improved liquid system method with the method of the prior art wherein the sample is immobilized on a solid support. They have shown that the liquid system outperforms the membrane-based assay in its ability to discriminate between cancer and non-cancerous samples.

One aspect of the invention is a method for detecting cancer or a precancerous condition in a subject, wherein a sample from the subject is assayed for the presence of a carbohydrate marker present in the sample associated with cancer or precancerous cells, comprising the steps:

(a) providing a sample from a subject;

(b) mixing the sample with an oxidation agent that is capable of oxidizing susceptible C-6 hydroxyl groups on the carbohydrate markers to aldehydes;

(c) adding an aldehyde detection agent to the mixture that produces a calorimetric change in the presence of an aldehyde; and

(d) detecting the calorimetric change in a liquid system, wherein the colorimetric change produced by the aldehyde detecting reagent is indicative of the presence of a carbohydrate marker associated with cancer or precancerous cells.

Another aspect of the invention is a method for detecting cancer or a precancerous condition in a subject, wherein a sample from the subject is assayed for the presence of a carbohydrate marker present in the sample associated with cancer or precancerous cells, comprising the steps:

(a) mixing the sample with an oxidation agent that is capable of oxidizing susceptible C-6 hydroxyl groups on the carbohydrate markers to aldehydes;

(b) adding an aldehyde detection agent to the mixture that produces a colorimetric change in the presence of an aldehyde; and

(c) detecting the calorimetric change in a liquid system, wherein the calorimetric change produced by the aldehyde detecting reagent is indicative of the presence of a carbohydrate marker associated with cancer or precancerous cells.

The term “subject” as used herein includes all members of the animal kingdom including human. The subject is preferably human.

The phrase “in a liquid system” as used herein means that the assay, particularly the detection of the calorimetric change, is conducted in a liquid phase and not on a solid support, such as a membrane.

The term “sample” as used herein refers to a fluid sample from a subject, including, without limitation, rectal mucus, saliva, lung sputum, breast nipple aspirate, cervical mucus, seminal fluid, plasma, blood serum and lymphatic fluid. The sample may be obtained from the subject by methods known to persons skilled in the art. For example, rectal or cervical mucus can be obtained by digital examination with a lubricated, gloved finger or suitable sampling device. The mucus is then recovered from the glove or device, for example with the aid of a solubilizing agent, preferably in a low volume to minimize sample dilution. The sample can also be extracted from a specimen sampling device. For example, the mucus sample can also be collected onto a swab (e.g. cotton, polyester, polyamide, foam, alginate). In one embodiment, the sample is extracted from the swab. Swabs constructed from calcium alginate are particularly suited for recovery of the sample due to salvation of the swab fibers in several reagents, including sodium citrate, glycerophosphate, sodium hexametaphosphate, sodium ethylene glycol-bis(2-aminoethylether)-N,N,N′,N′ tetraacetic acid (EGTA) and ethylenediaminetetraacetic acid (EDTA), preferably glycerophosphate, to form a clear gel or solution. In another embodiment, the sample is not extracted from the swab. Instead, the swabbed-sample is reacted directly with the oxidation agent, and then the aldehyde detecting agent in sufficient volume so that the liquid can be transferred to a vessel, such as a microwell, for analysis using a spectrophotometer or microplate reader.

In another example, lung sputum can be collected following deep inhalation and forceful coughing, but may require induction with hypertonic saline, such as ≧3% NaCl.

The sample may require processing prior to using with the method of the invention. For example, sputum is generally immiscible with aqueous reagents and thus precludes direct reactivity with galactose oxidase and Schiff's reagent unless the gel-like matrix is first disrupted and the gel liquefied. The mucous samples can be easily liquefied using different methods and classes of agents including mechanical degradation and high-frequency oscillations, reducing agents, charged oligosaccharides (dextran, heparin), sodium chloride, or enzymes (DNase, gelsolin). Commonly used reducing agents such as N-acetylcysteine (NAC), β-mercaptoethanol (β-ME), dithiothreitol (DTT) and phosphines, which cleave disulphide bonds, are particularly effective mucolytics. A particularly effective disulfide-cleaving reagent to liquefy sputum prior to assay with the method of the invention is tris(2-carboxyethyl)phosphine (TCEP). TCEP is an alkyl derivative of phosphine and is highly specific. It is both stable and odorless.

Accordingly, in one embodiment of the invention, the sample is liquefied prior to mixing with the oxidation agent.

It may also be advantageous to remove cellular material and particulates from the sample prior to mixing the sample with the oxidation agent. For example, the sample may be centrifuged or filtered to remove cells or cellular material.

The method of the invention is directed at detecting cancer or a precancerous condition in a subject wherein the sample is assayed for the presence of a carbohydrate marker associated with cancer or precancerous cells. Accordingly, the term “cancer or precancerous cells” as used herein includes any cancer or precancerous cells that expresses a carbohydrate marker detectable using an oxidation reagent, such as galactose oxidase, and an aldehyde detection agent, such as Schiff's reagent. The carbohydrate marker associated with the cancer or precancerous cells may be present on the surface of the cells or may be produced by the cells in a soluble form. The method of the invention does not require that the cancer or precancerous cells be present in the sample. For example, the method of the invention can detect carbohydrate markers which are soluble or membrane-associated but free of the cells.

The phrase “associated with cancer or precancerous cells” as used herein means that the carbohydrate marker is expressed by or present on cancer or precancerous cells in higher amounts as compared to non-cancer or non-precancerous cells. Thus, there are higher amounts of the carbohydrate marker in samples from subjects with cancer or a precancerous condition as compared to subjects without cancer or a precancerous condition.

In one embodiment of the invention, cancer includes, without limitation, cervical cancer, uterine cancer, ovarian cancer, pancreatic cancer, kidney cancer, gallbladder cancer, liver cancer, head and neck cancer, gastrointestinal cancer, breast cancer (such as carcinoma, ductal, lobular, and nipple), prostate cancer, testicular cancer, oral cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, sarcomas, colon cancer, rectal cancer, stomach cancer, bladder cancer, pancreatic cancer, endometrial cancer, plasmacytoma, lymphoma, and melanoma. In a preferred embodiment, the cancer includes, without limitation, colon cancer, rectal cancer, oral cancer, lung cancer, breast cancer, vaginal cancer, cervical cancer, ovarian cancer, endometrial cancer, prostate cancer and hematologic cancer.

The method of the invention detects carbohydrate markers on mucin glycoproteins that are associated with cancer or precancerous lesions. The carbohydrate markers include without limitation D-galactose (Gal), N-acetyl-D-galactosamine (GalNAc), D-galactose-β-[1→3]-N-acetyl-D-galactosamine (GalGalNAc), also known as Thomsen-Friedenreich (TF) or T-antigen, Fuc-α-1→2-Gal-β-(1→4)-Fuc-α-1→3-GlcNAc, Fuc-α-1→2-Gal-β-(1→4)-Fuc-α-l→3-GlcNAc-β-(1→3)-Gal-β-(1→4)-GlcNAc, and Fuc-α-l→2-Gal-β-(1→4)-Fuc-α-1→3-GlcNAc-β-(1→3)-Gal-β-(1→4)-Fuc-α-1→3-GlcNAc.

According to the method of the invention, the sample from the subject is mixed with an oxidation agent that is capable of oxidizing susceptible hydroxyl groups (carbon 6 primary alcohol) on the carbohydrate markers to aldehydes. In one embodiment of the invention, the oxidation agent is galactose oxidase.

The method of the invention also requires the use of an aldehyde detection agent. Specifically, an aldehyde detection agent is added to the mixture of the sample and oxidation agent, and produces a colorimetric change in the presence of an aldehyde. The aldehyde detection agent includes, and is not limited to, basic fuchsin, such as Schiff's reagent, and Glycoprotein Detection Reagent, Product Code 23262 (Pierce Biotechnology, Inc.), which forms a magenta color in the presence of an aldehyde group. The aldehyde detection agent is preferably storage stable as described in U.S. Pat. No. 5,348,860.

In order to know that the reagents are working and to minimize the potential for false-negative results due to inactivation or premature deterioration of the components of the method, a positive control can be used. Positive controls include, and are not limited to, carbohydrates that are reactive with the oxidation agents and aldehyde detecting agents of the invention, such as Gal, GalNAc, GalGalNAc, Fuc-α-1→2-Gal-β-(1→4)-Fuc-α-1→3-GlcNAc, Fuc-α-1→2-Gal-β-(1→4)-Fuc-α-l→3-GlcNAc-β-(1→3)-Gal-β-(1→4)-GlcNAc, and Fuc-α-l→2-Gal-β-(1→4)-Fuc-α-1→3-GlcNAc-β-(1→3)-Gal-β-(1→4)-Fuc-α-1→3-GlcNAc. As such, positive controls include, without limitation, glycoproteins or high-molecular weight mucins (e.g., porcine gastric mucin) and polysaccharides (e.g., guar).

In one example, guar is used as a positive control. Guar is a water-soluble, high-molecular weight carbohydrate polymer (galactomannan) comprising a mannose backbone with randomly-spaced galactose side chains. The average ratio of gal:mannose is 1:2 for a molecular composition of galactose of 30%. Unlike some proteins, including mucins such as porcine gastric mucin, guar is non-reactive with Schiff's unless oxidized first with galactose oxidase. Hence, background noise is negligible and signal:noise ratio high at various concentrations. Guar serves as an ideal positive control for both galactose oxidase and Schiff's reagent. Preformed aldehydes such as acetaldehyde, formaldehyde, or glutaraldehyde, can be used to monitor the activity of Schiff's or other aldehyde-detecting reagents.

The method of the invention has the advantage over the prior art in that it does not depend on a subjective visualization or sophisticated instrumentation for quantifying color attributes for appraisal of color which is developed in the sample by treatment with the oxidation agent, such as galactose oxidase, and the aldehyde detection agent, such as Schiff's reagent. In contrast, the method of the invention allows an objective measure based on simple absorption at a predefined wavelength using standard laboratory spectrophometers or microplate readers. A person skilled in the art will appreciate that the predefined wavelength to measure absorption will depend on composition of the sample being analyzed, including the type of sample, the type of oxidation agent, the type of aldehyde detection agent, and any other reagents used to store or process the sample, such as a solubilizing agent, liquefying agent or solvent. For example, using galactose oxidase and Schiff's reagent, the calorimetric change can be relatively quantified using a spectrophometer or microplate reader between 530-570 nm. Typically, spectrophometers or microplate readers are capable or reading within the visible spectrum, i.e. 440-700 nm. Thus, 550 nm or an appropriate wavelength at or near the maximum absorption for the detector-aldehyde complex can be used to detect the calorimetric change. The method of the invention also has the advantage that it is amenable to automation and high-throughput batch processing using liquid handling systems.

The invention also includes a kit for detecting cancer or a precancerous condition in a subject, comprising an oxidation agent and an aldehyde detection agent, and instructions for carrying out the method of the invention.

In one embodiment, the oxidation agent is galactose oxidase and the aldehyde detection agent is basic fuchsin, such as Schiff's reagent. Preferably, the basic fuchsin is storage stable.

The kit can also include a reducing agent to liquefy the sample, such as NAC, β-ME, DTT or TCEP. In addition, the kit can include a filter to remove cellular materials and particulates from the sample. The kit can also include a positive control, such as guar.

The following non-limiting examples are illustrative of the present invention:

### EXAMPLES

### Example 1

### Rectal Mucus

Rectal mucus is obtained by digital examination with a lubricated, gloved finger. For evaluation of the reactivity of said mucus with GOS, immobilisation onto a water-insoluble substrate (e.g., membrane filter, glass slide) has previously been required. For a liquid-phase GOS assay, mucus is recovered from the glove, with the aid of a solubilizing agent, preferably in a low volume to minimize sample dilution. Alternatively, the mucus specimen can be collected onto a swab (cotton, polyester, polyamide, foam, alginate), extracted and subsequently tested with GOS in solution. Swabs constructed from calcium alginate are particularly suited for recovery of sample due to salvation of the swab fibers in several reagents (sodium citrate, glycerophosphate, sodium hexametaphosphate, EGTA or EDTA) to form a clear gel or solution. Mucus released into the gel/sol can be tested with GOS as follows:
1. Incubate aliquot of solubilized alginate swab containing rectal mucus specimen (some cross-linked mucus specimens may require treatment with disulphide-breaking reagent and/or adjustment of concentration by dilution) with GO to allow oxidation of C6 hydroxyl groups on susceptible sugars to aldehydes.
2. Incubate above reaction mixture with Schiff's reagent to permit formation of colored adduct with aldehydes.
3. Read absorbance of above reaction product at 550 nm.

### Example 2

### Saliva and Lung Sputum

Saliva may be collected in a cup and is sufficiently fluid for pipetting without pre-treatment with a solubilising or mucolytic agent. Saliva can be freed of host buccal cells and bacterial cells by centrifugation prior to pipetting for assay. Saliva obtained with expectorated sputum can be separated by centrifugation. Saliva can be processed with GOS directly.

Sputum is a thick, gel-like respiratory secretion containing mucin macromolecules (high-molecular weight glycoproteins), bacterial polysaccharides and genetic material, host leukocyte DNA and actin filaments, as well as normal and abnormal pulmonary cells. Sputum is frequently expectorated with saliva and can be isolated by centrifugation. Sputum is typically collected spontaneously following deep inhalation and forceful coughing but may require induction with hypertonic saline (e.g., ≧3% NaCl). The consistency of sputum renders it immiscible with aqueous reagents. This precludes direct reactivity with GOS unless the gel-like matrix is first disrupted and the gel liquefied and turned into a solution. Sputum and other mucous samples are easily liquefied using techniques known to persons skilled in the art. The three-dimensional structure forming the viscous gel is due to molecular interactions involving various types of bonds (covalent, ionic, hydrogen, van der Waals forces). As a result, different methods and classes of agents have been used to liquefy mucous gels: mechanical degradation and high-frequency oscillations, reducing agents, charged oligosaccharides (dextran, heparin), sodium chloride, enzymes (DNase, gelsolin). Commonly used reducing agents such as NAC, β-ME, DTT and phosphines, which cleave disulphide bonds, are particularly effective mucolytics. A particularly effective disulfide-cleaving reagent to liquefy sputum prior to assay with GOS is TCEP. TCEP is an alkyl derivative of phosphine and is highly-specific. It is both stable and odorless. The GOS assay is carried out as follows:
1. Sputum is separated from saliva by centrifugation.
2. Saliva is decanted, and an aliquot of sputum treated with TCEP and allowed to incubate at ambient temperature.
3. Saliva and liquefied sputum are incubated separately with GO to permit oxidation of C6 hydroxyl groups on susceptible sugars to aldehydes.
4. To these individual mixtures are added Schiff's reagent which forms colored adducts with the aldehyde groups.
5. The absorbance of the solutions is measured at 550 nm.
   The intensity of color is proportional to the amount of tumor marker present in the specimen. A cutoff, derived from the reactivity of saliva or sputa obtained from apparently healthy individuals (no pulmonary pathology) and patients with benign lung disease, is used to define a negative test result, thereby allowing detection of the presence of lung cancer or a pre-cancerous lesion.

### Example 3

### Breast Nipple Aspirates

Breast nipple aspirate fluid (NAF) may be clear, slightly cloudy and/or discolored. It is amenable to pipetting and NAF can therefore, be tested directly by the GOS procedure without initial manipulation with disulphide reducing agents. The method is, in principle, essentially the same as for rectal mucus, saliva and lung sputum, but adjusted for specimen and reagent volumes.

### Example 4

### Other Secretions

Mucous secretions from the vagina (endometrium, cervix) or prostate (seminal fluid) are also candidates for testing by GOS in liquid phase. Endometrial or cervical mucus collected onto a swab can be tested directly or may require extraction prior to GOS testing. Seminal fluid, cervical or endometrial mucus may first require manipulation (centrifugation) to remove cellular material (sperm, prostate, cervical or endometrial cells) and/or pretreatment with mucolytics. The GOS procedure is identical to that described for rectal mucus, saliva, lung sputum and NAF.

### Example 5

### Blood

Blood fractionated into plasma (non-cellular portion) and serum (non-cellular portion minus clotting factors/proteins) provides an aqueous environment for the measurement of various analytes, including GOS-reactive markers. Plasma or serum, suitably diluted, can be treated with GO and subsequently Schiff's reagent, without pre-incubation with disulphide-reducing agents.

### Example 6

### Reagent Positive Control

It is advantageous to know that reagents are working as anticipated, within guidelines or specifications, at the time of assay to ensure adequate potency for detection of low levels of analyte/marker and to minimize potential for false-negative results due to inactivation or premature deterioration (inappropriate storage or exposure to environmental conditions, contamination) of components. Guar is a water-soluble, high-molecular weight carbohydrate polymer (galactomannan) comprising a mannose backbone with randomly-spaced galactose side chains. The average ratio of gal:mannose is 1:2 for a molecular composition of galactose of 30%. Unlike some proteins, including mucins such as porcine gastric mucin, guar is non-reactive with Schiff's unless oxidized first with GO. Hence, background noise is negligible and signal:noise ratio high at various concentrations. Guar serves as an ideal positive control for both GOS reagents.

### Example 7

### Comparison of Liquid-Phase GOS Assay with Membrane GOS Assay

A. Study Sample

Membrane and liquid-phase GOS assays were compared in frozen, banked lung sputa obtained from a local hospital. Twenty specimens comprising 5 from normal subjects (no pulmonary pathology), 1 from an apparently healthy smoker, 2 from patients identified as having benign lung disease (BLD), and 12 from patients diagnosed with early-stage lung cancer (8 Stage I, 4 Stage II), were tested in parallel in the membrane and liquid-phase GOS assays after processing with mucolytic.

B. Sputum Processing

An aliquot of saliva-free sputum (approximately 0.25 g), was thawed and mixed with an equal volume by weight, of 50 mM disulphide reducing agent, tris(2-carboxyethyl)phosphine (TCEP) in a 1.5-mL microfuge tube. After vigorous vortexing for 15 seconds (S/P® Vortex Mixer, Baxter Diagnostics Inc., setting 10), the mixture was allowed to incubate for 60 minutes on a shaking platform (geljiggler, Interface Systems; maximum speed). The liquefied sputum was vortexed, centrifuged for 10 minutes at 10,000 rpm (Eppendorf 5415C centrifuge, Brinkmann Instruments, Inc.) and the supernatant fluid separated from the pellet.

C. Membrane GOS Assay

Processed (liquefied) sputum (20 μL) was spotted, in duplicate, onto a glass fiber membrane (954-AH, Whatman Inc.) affixed to a polypropylene support (TapeTest Device, IMI International Medical Innovations Inc.) via 3M 9877 double-sided adhesive and allowed to air-dry overnight (16-20 hr) at ambient temperature. GOS reagents were equilibrated to room temperature before use. Sputum spots were incubated with an equal volume of GO at 100 U/mL for 10 minutes. The device was transferred to a Coplin jar and rinsed for 1 minute with de-ionized water. The membrane was drained of excess water, incubated for 1 minute with 1 mL Schiff's reagent then washed 4 times for 10 minutes in tap water. The membrane was air-dried overnight and developed color read with a reflectance spectrophotometer (X-Rite, Inc.).

D. Liquid-Phase GOS Assay

Duplicate aliquots of processed (liquefied) sputum (50 μL) were incubated in microtubes with an equal volume of GO (100 U/mL) for 30 minutes at ambient temperature on the shaking platform. Schiff's reagent (50 μL) was added and the mixtures incubated a further 30 minutes while shaking. A 100-μL sample of the final reaction mixture was transferred to a round-bottom microwell (VWR International) and the absorbance at 550 nm read in a microplate reader (Bio-Tek EL800).

Results

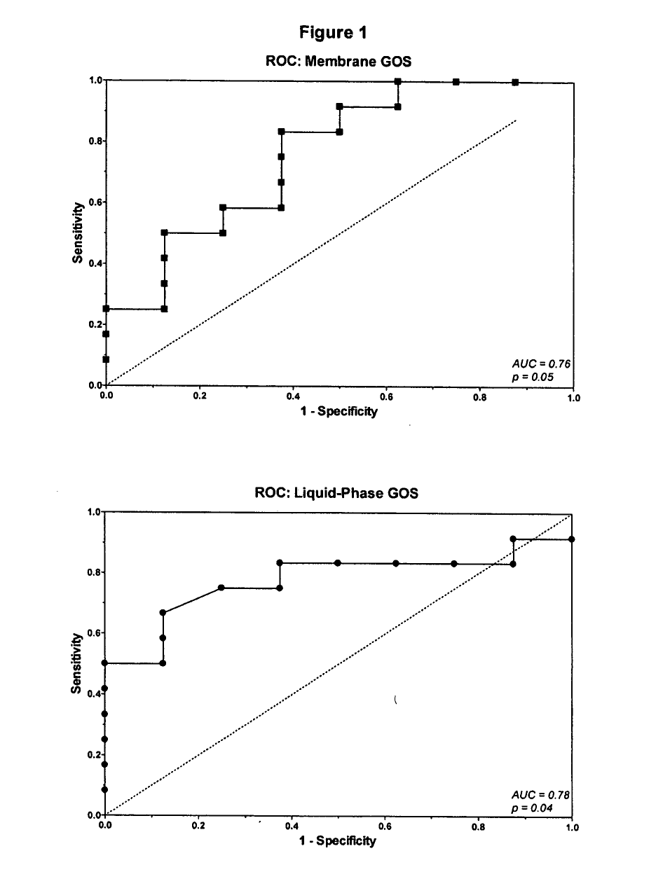

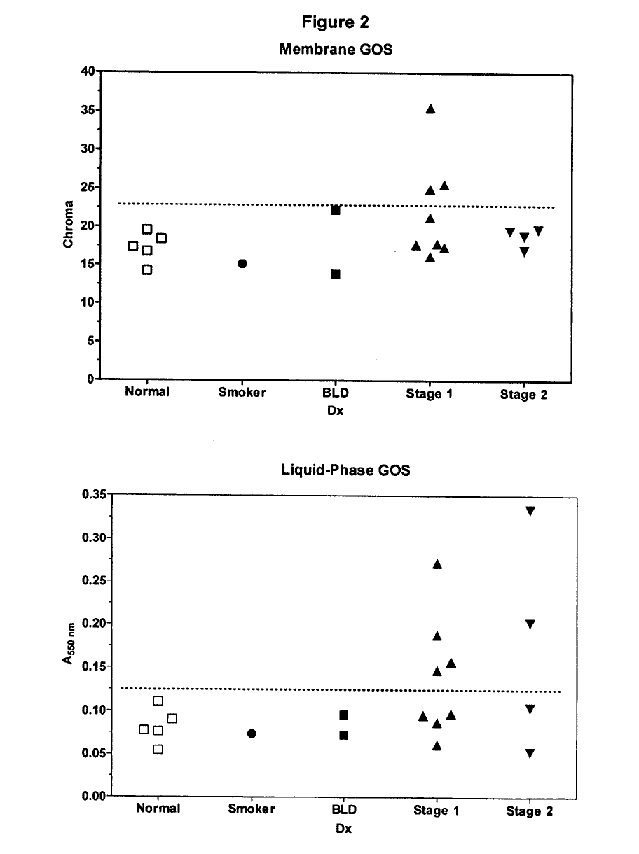

The reaction product of the membrane GOS test on sputa was analyzed by examining both chroma and hue, 2 attributes of color. Hue represents the perceived color and is described in numerical terms (degrees) as the position of the colors of the visible spectrum on a color wheel (A Guide to Understanding Color Communication, X-Rite, Inc.). Chroma or saturation is a measure of the vividness or dullness of hue. Low chroma values are indicative of the latter (greyer in appearance) whereas high values indicate the hue is closer to the pure color. Chroma may be more informative and discriminating when the colored products exhibit a narrow range in hue. ROC (Receiver-Operator Characteristic) analysis (a measure of the clinical performance of a test) of hue in the membrane GOS assay revealed an area under-the-curve (AUC) equal to 0.57 (0.50 denotes a test with equal sensitivity and specificity; 1.00 is a perfect test with no false positives or negatives) that was statistically not significant (p=0.59), indicating that hue had little/no capacity for differentiating cancer from non-cancerous cases (normals, BLDs and smoker) in this study sample. The ROC curve for chroma was better (FIG. 1, top panel) and had an AUC=0.76 that approached statistical significance (p=0.05, where p<0.05 is generally considered to be significant). However, at a cutoff intended to exclude subjects with no clinical evidence of cancer (i.e., >22.5; FIG. 2, top panel), the membrane GOS assay would detect only 3 of 12 cancers (25% sensitivity). By contrast, the ROC curve for the liquid-phase GOS assay had a different shape, produced an AUC (0.78) that was similar to that of chroma in the membrane assay but, that was statistically significant (p=0.04), implying some discriminatory ability between diseased and non-diseased individuals (FIG. 1, bottom panel). At a cutoff=0.125 absorbance units (550 nm), this assay correctly identified 6/12 (i.e., 2-fold improvement in sensitivity) sputa from lung cancer patients with no false-positives (FIG. 2, bottom panel). Thus, in this study sample of 20 sputa, the liquid-phase GOS assay outperformed the membrane-based assay. Modification or refinement of the solution assay through adjustment of one or more assay parameters could result in additional enhancement of the liquid GOS test.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

We claim:

1. A method for detecting cancer or a precancerous condition in a subject wherein a sample from the subject is assayed for the presence of a carbohydrate marker present in the sample associated with cancer or precancerous cells, comprising the steps:
(a) mixing the sample with an oxidation agent that is capable of oxidizing susceptible C-6 hydroxyl groups on the carbohydrate markers to aldehydes;
(b) adding an aldehyde detection agent to the mixture that produces a calorimetric change in the presence of an aldehyde; and
(c) detecting the colorimetric change in a liquid system, wherein the calorimetric change produced by the aldehyde detecting reagent is indicative of the presence of a carbohydrate marker associated with cancer or precancerous cells.

2. The method according to claim 1, wherein the oxidation agent is galactose oxidase.

3. The method according to claim 1, wherein the carbohydrate marker comprises D-galactose (Gal), N-acetyl-D-galactosamine (GalNAc), D-galactose-β-[1→3]-N-acetyl-D-galactosamine (GalGalNAc), Fuc-α-1→2-Gal-β-(1→4)-Fuc-α-1→3-GlcNAc, Fuc-α-1→2-Gal-β-(1→4)-Fuc-α-l→3-GlcNAc-β-(1→3)-Gal-β-(1→4)-GlcNAc, and Fuc-α-l→2-Gal-β-(1→4)-Fuc-α-1→3-GlcNAc-β-(1→3)-Gal-β-(1→4)-Fuc-α-1→3-GlcNAc.

4. The method according to claim 1, wherein the aldehyde detection agent is basic fuchsin.

5. The method according to claim 4, wherein the basic fuchsin is Schiff's reagent.

6. The method according to claim 1, wherein the calorimetric change is quantified spectrophotometrically by determining the absorbance of the sample at a wavelength between about 530 nm to about 570 nm.

7. The method according to claim 1, wherein the colorimetric change is quantified spectrophotometrically by determining the absorbance of the sample at a wavelength about 550 nm.

8. The method according to claim 1, wherein the sample is liquefied prior to mixing the sample with the oxidation agent.

9. The method according to claim 8, wherein the sample is liquefied using a reducing agent.

10. The method according to claim 9, wherein the reducing agent is N-acetyl cysteine, β-mercaptoethanol, dithiothreitol or tris(2-carboxyethyl)phosphine.

11. The method according to claim 8, wherein the sample is liquefied using mechanical degradation or high-frequency oscillations.

12. The method according to claim 1, wherein cellular material and particulates are removed from the sample prior to mixing the sample with the oxidation agent.

13. The method according to claim 12, wherein the cellular materials and particulates are removed from the sample by centrifugation or filtration.

14. The method according to claim 1, wherein the sample is extracted from a specimen sampling device.

15. The method according to claim 1, wherein a positive control is used.

16. The method according to claim 15, wherein the positive control is guar.

17. A kit for detecting cancer or a precancerous condition in a subject, comprising an oxidation agent and an aldehyde detection agent, and instructions for carrying out the method according to claim 1.

18. The kit according to claim 17, wherein the oxidation agent is galactose oxidase.

19. The kit according to claim 17, wherein the aldehyde detection agent is basic fuchsin which is storage stable.

20. The kit according to claim 19, wherein the basic fuchsin is Schiff's reagent.

21. The kit according to claim 17, which further comprises a filter to remove cellular materials and particulates from the sample.

22. The kit according to claim 17, which further comprises a reducing agent to liquefy the sample.

23. The kit according to claim 22, wherein the reducing agent is N-acetyl cysteine, β-mercaptoethanol, dithiothreitol or tris(2-carboxyethyl)phosphine.

24. The kit according to claim 17, which further comprises a positive control.

25. The kit according to claim 24, wherein the positive control is guar.
